(19) United States
(12) Patent Application Publication
(10) Pub. No.: US 2017/0059561 A1
(43) Pub. Date: Mar. 2, 2017

(54) Thermally Stable Electrochemical Sensor With Long Shelf-Life

(71) Applicants: Shekhar BHANSALI, Weston, FL (US); Aparajita SINGH, Miami, FL (US); Syed Khalid PASHA, Miami, FL (US)

(72) Inventors: Shekhar BHANSALI, Weston, FL (US); Aparajita SINGH, Miami, FL (US); Syed Khalid PASHA, Miami, FL (US)

(73) Assignee: The Florida International University Board of Trustees, Miami, FL (US)

(21) Appl. No.: 15/247,460

(22) Filed: Aug. 25, 2016

Related U.S. Application Data
(60) Provisional application No. 62/211,172, filed on Aug. 28, 2015.

(51) Int. Cl. G01N 33/543; G01N 27/327

(52) U.S. Cl. CPC G01N 33/5438; G01N 33/54306; G01N 27/3271; G01N 2610/00; G01N 2469/10

(57) ABSTRACT

The subject invention provides the materials and methods for fabricating and using an electrochemical immunosensing device to detect a target antigen. In a preferred embodiment, the subject invention utilizes Camelid-derived, single-domain antibodies as the sensing agents immobilized onto the surface of the immunosensing device, more specifically, the working electrode of the immunosensing device. Furthermore, embodiments of the subject invention provide means for increasing the device's detection sensitivity by utilizing a working electrode optionally configured with an array of interdigitated electrodes.

### CROSS-REFERENCE TO A RELATED APPLICATION

This application claims the benefit of U.S. Provisional Application Ser. No. 62/211,172, filed Aug. 28, 2015, which is incorporated herein by reference in its entirety.

This invention was made with government support under contract number EEC-1160483 awarded by the National Science Foundation. The government has certain rights in the invention.

### BACKGROUND OF INVENTION

Electrochemical immunosensing is based on the principle of measuring the changes in electrical properties of a conductive substrate as a result of the addition of an adsorbate at the surface (A. Kaushik, A. Vasudev, S. K. Pasha and S. Bhansali, Biosensors and Bioelectronics, 53, 499 (2014); Y. Wan, Y. Su, X. Zhu, G. Liu and C. Fan, Biosensors and Bioelectronics, 47, 1 (2013); A. Kaushik, S. K. Arya, A. Vasudev and S. Bhansali, Journal of Nanoscience Letters, 3, 32 (2013)). Examples of adsorbates provided herein include SAMs, antibody layerS, and target antigens. The change in electrical signal is attributed to changes in the concentration of the electroactive reduction-oxidation (redox) species at the electrode's surface. The magnitude of the redox current decreases with each additional layer of adsorbates. This is due to the fact that these adsorbates, generally being organic and/or biological species, are non-conductive and, thus, hinder the electron transport at the surface of the electrode as shown in FIG. 3.

Electrochemical immunosensing has demonstrated success in large-scale production and for long-term operations among label-free sensors. The processing capability of the microfabrication industry has enabled production of highly sensitive electrodes with low detection limits. The simplicity of electronic circuitry for electrochemical detection and inexpensive high-volume manufacturing has driven efforts to bring electrochemical immunosensing up to speed with other immunosensing techniques (C. Loncaric, Y. Tang, C. Ho, M. A. Parameswaran and H.-Z. Yu, Sensors and Actuators B: Chemical, 161, 908 (2012); P. B. Lillehoj, M.-C. Huang, N. Truong and C.-M. Ho, Lab on a Chip, 13, 2950 (2013)).

Electrochemical sensing has advantages over other analyte detection techniques such as chromatography, radioimmunoassay (RIA), electro-chemiluminescence immunoassay (ECLIA), enzyme-linked immunosorbent assay (ELISA), surface plasmon resonance (SPR), and quartz crystal microbalance (QCM). For these techniques, the turn-around time from sampling to results is typically from days to weeks, and they often involve sending samples to a diagnostic laboratory.

Over the years, electrochemical sensing has satisfied the need of sensing platforms that can be deployed at point-of-care (POC) sites in order to quantify analytes instantaneously in relevant samples. In addition, electrochemical biosensors have addressed issues such as large sample volume requirement and high cost related to conventional laboratory-based diagnostic methods.

Nonetheless, a number of challenges still remain in the evolving field of electrochemical biosensors. One such challenge pertains to the stability of electrochemical sensors when subjected to long storage time and when subjected to harsh environmental factors. Other shortcomings, such as batch-to-batch inconsistency of polyclonal antibodies and extensive production time of monoclonal antibodies, pose additional limitations.

Advantageously, camelid-derived antibodies are reported to be inherently unaffected by changes in temperature and retain their structure due to high refolding efficiency (E. M. Clingerman and A. Brown, Biological Research for Nursing, 14, 27 (2012); A. Cecchi, M. Rovedatti, G. Sabino and G. Magnarelli, Ecotoxicology and Environment Safety, 80, 280 (2012); Goldman et al., 2006, Anal. Chem., 2006, 78 (24), pp 8245-8255). Successful utilization of these antibodies has been demonstrated effective in detecting various pathogens and toxins (G. P. Anderson, J. L. Liu, M. L. Hale, R. D. Bernstein, M. Moore, M. D. Swain and E. R. Goldman, Analytical Chemistry 80 (24), 9604-9611 (2008); G. P. Anderson, R. D. Bernstein, M. D. Swain, D. Zabetakis and E. R. Goldman, Analytical Chemistry 82 (17), 7202-7207 (2010)).

### BRIEF SUMMARY

The subject invention provides materials and methods of fabricating and using electrochemical immunosensing devices to detect a target antigen. In a preferred embodiment, the subject invention utilizes camelid-derived, single-domain antibodies as the sensing agents immobilized onto a surface of an immunosensing device. In a specific embodiment, the camelid-derived antibody is immobilized onto the surface of the working electrode of the immunosensing device. Further, embodiments of the subject invention provide means for increasing the device's detection sensitivity by utilizing a working electrode configured with an array of interdigitated electrodes.

Advantageously, the technologies provided herein improve the stability of electrochemical immunosensing devices at temperatures above room temperature, therefore capable of extending the devices' shelf-life and facilitating reliable point-of-care deployment in a wide variety of settings.

In one aspect, the subject invention provides a method of fabricating an electrochemical immunosensing substrate, the method comprising configuring the surface of a conductive substrate with an array of interdigitated electrodes and chemically immobilizing a layer of single domain antibodies onto the surface of the substrate. In certain embodiments, the immunosensing substrate is the working electrode of an immunosensing device. The device may further comprise a counter electrode and a reference electrode.

In certain embodiments, the subject invention provides detection devices that can be worn by a subject. The subject may be, for example, a person or animal. Advantageously, these devices can function for extended periods of time and even in harsh environments.

In some embodiments, the conductive electrode is chemically functionalized prior to the immobilization of the thermally stable antibodies. In a preferred embodiment, the chemical functionalization comprises at least one self-assembled monolayer.

In some embodiments, the theunally stable antibodies are single-domain antibodies. Preferred embodiments provide that the single-domain antibodies are derived from species belonging to the Camelidae family.

In another aspect, the subject invention provides a method for detecting a target antigen, comprising:

a) providing an immunosensing substrate, the surface of the substrate being configured to have an array of interdigitated electrodes and chemically immobilized with a layer of antibodies;

b) contacting the substrate with a sample;

c) applying a voltage to the substrate; and

d) monitoring a change in current response of the substrate as antigens bind with the immobilized antibodies.

In some embodiments of the invention, the electrochemical response of the device is measured by monitoring the redox current output of the electrode as a function of varying the applied voltage using cyclic voltammetry (CV). In certain embodiments, a reduction-oxidation (redox) probing moiety is employed, and preferably comprises a combination of ferricyanide and ferrocyanide anions in salt forms such as, for example, potassium salts.

In some embodiments, the application of voltage to the electrode is cyclic, ranging from about −1.0 V to about 1.0 V. Exemplary embodiments provide that the voltage is cycled between about −0.6 V to about 0.6 V.

In certain embodiments of the subject invention, the antibody-antigen binding activity, evaluated as the change in current response to the applied voltage in comparison with a neat substrate, remains effectively constant for 7 days or more at temperatures of about 40° C. or higher.

The term “neat substrate” herein refers to a conductive substrate, optionally configured with an array of interdigitated electrodes, not comprising any surface modifications or antibodies.

In an exemplary embodiment, the target antigen is ricin-chain A, derived from a castor oil plant Ricinus communis. Its complementary single-domain antibody is hereafter denoted ricin_{sdAb}.

In yet another aspect, the subject invention provides an electrochemical immunosensing substrate, whose surface is configured to comprise an array of interdigitated electrodes and chemically immobilized with a layer of antibodies.

In certain embodiments, the immunosensing substrate is the working electrode of an immunosensing device, which may further comprise a counter electrode and a reference electrode.

In certain embodiments, the working electrode is optionally functionalized by at least one self-assembled monolayer prior to the immobilization of the antibodies.

In an exemplary embodiment, the device is stable at temperatures between room temperature and about 40° C. for up to about 7 days.

### BRIEF DESCRIPTION OF THE DRAWINGS

FIG. 1A shows the structure of a conventional antibody with its antigen binding fragment, the single chain antibody (scFv).

FIG. 1B shows the structure of an unconventional heavy chain antibody (VHH) isolated from members of the Camelidae family with its antigen binding fragment (sdAb).

FIG. 2 shows an exemplary process of fabrication of interdigitated electrodes (IDEs) that involves: (i) fabrication of gold IDEs, (ii) immobilization of DTSP-SAM on the gold IDEs, (iii) immobilization of ricin_{sdAbs}, and (iv) electrochemical detection of an antigen such as ricin chain-A.

FIG. 3 shows a schematic of the working principle of a redox probing moiety in detecting antigen binding activity of the immunosensing device. Note that the decrease in peak intensity indicates the addition of each adsorbing layer.

FIG. 4 shows cyclic voltammetry (CV) measurements of the gold electrode successively adsorbed with a self-assembled monolayer (SAM), camelid-derived sdAbs, and ricin-chain A antigens.

FIG. 5 shows the comparison of CV responses of sdAbs stored at 40° C. at time 0 and after 24 hours elapsed.

FIG. 6 shows the comparison of CV responses between the electrode adsorbed with sdAbs, sdAbs bound with the antigens at room temperature, and sdAbs bound with the antigens stored at 40° C. for 7 days.

FIG. 7 shows the comparison of CV responses of the electrode adsorbed with sdAbs at 55° C. between time points 0, 24 hours, 48 hours, and 72 hours.

FIG. 8 demonstrates the effect of pH on the electrochemical response of an exemplary embodiment of the immunoelectrode provided herein.

FIG. 9 shows the CV response that indicates ricin polyclonal antibodies are not stable in a 24-hour period.

FIG. 10 shows the CV response that indicates ricin monoclonal antibodies are not stable in a 24-hour period.

FIG. 11 shows the EIS measurements of an exemplary immunoelectrode with various adsorbates at the surface: (i) only gold electrode, (ii) with SAM layer, (iii) with ricin_{sdAb}, and (iv) with ricin-chain A antigen.

FIG. 12 shows a calibration curve obtained from the electrochemical response of an exemplary ricin_{sdAb}/DTSP-SAM/IDEs immunoelectrode as a function of ricin-chain A concentration (1 log(fg/mL)−log (μg/mL)).

FIG. 13A shows the time-dependent organization of an exemplary SAM.

FIG. 13B shows an ordered SAM with ricin_{sdAb}.

FIG. 13C shows a disordered SAM with ricin_{sdAb}.

FIG. 13D shows an ordered SAM with tilted or disoriented ricin_{sdAb}.

FIG. 13E shows a disordered SAM with tilted ricin_{sdAb}. Possible reasons for increased blockage to the sensor surface with rise in temperature arise from adsorption of SAMs such as those depicted in (FIG. 13C), (FIG. 13D), and (FIG. 13E).

### DETAILED DISCLOSURE

The subject invention provides materials and methods of fabricating and using an electrochemical immunosensing device to detect a target antigen. In a preferred embodiment, the subject invention discloses the use of camelid-derived, single-domain antibodies as the sensing agents immobilized onto the surface of the immunosensing device, more specifically, the working electrode of the immunosensing device. In certain embodiments, the device's detection sensitivity is increased by utilizing a working electrode configured with an array of interdigitated electrodes.

Advantageously, the technologies provided herein improve the stability of electrochemical immunosensing devices at temperatures above room temperature, therefore capable of extending the devices' shelf-life for reliable point-of-care deployment.

### Definitions

The term “antibody” refers to an immunoglobulin molecule that recognizes and specifically binds to a target, such as a protein, polypeptide, peptide, carbohydrate, polynucleotide, lipid, or combinations thereof through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), camelid-derived heavychain polypeptides (VHH) and fragments thereof (e.g., truncated VHH), single chain Fv (scFv) and mutants thereof, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, and radioisotopes.

A “variable region” of an antibody refers to the variable region of the antibody light chain or the variable region of the heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies.

The term “antibody fragment” refers to a portion of an intact antibody and refers to the antigenic-determining variable regions of an intact antibody. Examples of antibody fragments include, but are not limited to, camelid-derived VHH polypeptide fragments (e.g., truncated VHH), Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to “polyclonal antibodies” that typically include different antibodies directed against different antigenic determinants. The term “monoclonal antibody” encompasses both intact and full-length monoclonal antibodies as well as antibody fragments (such as Fab, Fab′, F(ab′)2, Fv), camelid-derived VHH polypeptides and fragments thereof (e.g., truncated VHH), single chain Fv (scFv) and mutants thereof, fusion proteins comprising an antibody portion, and any other modified immunoglobulin molecule comprising an antigen recognition site. Furthermore, “monoclonal antibody” refers to such antibodies made by any number of manners including, but not limited to, hybridoma, phage selection, recombinant expression, and transgenic animals.

The term “single-domain antibody” (sdAb) refers to an antibody whose complementary determining regions are part of a single-domain polypeptide. Examples include, but are not limited to, heavy chain antibodies, antibodies naturally devoid of light chains, single domain antibodies derived from conventional 4-chain antibodies, and recombinant antibodies. Single-domain antibodies may be any of what the current state of the art provides, or any future single-domain antibodies not yet realized. Single-domain antibodies may be derived from any species, including, but not limited to, mouse, human, camel, llama, goat, rabbit, and bovine. In a specific embodiment of the subject invention, the single-domain antibodies are derived from heavy chain antibodies of species belonging to the Camelidae family, including, but not limited to, camel, llama, dromedary, alpaca, and guanaco.

The term “antigen” as used herein refers to any substance capable of eliciting an immune response when introduced into a subject. An immune response includes, for example, the formation of antibodies and/or cell-mediated immunity. Non-limiting examples of antigens include, but are not limited to, infectious agents (e.g., bacteria, viruses, fungi, prion, parasite, and the like), polypeptides (e.g., proteins, including viral proteins), polynucleotides (e.g., DNA, RNA), cells, and compositions containing antigens.

In one aspect, the subject invention provides a method of fabricating an electrochemical immunosensing substrate, the method comprising configuring the surface of a conductive substrate with an array of interdigitated electrodes and chemically immobilizing a layer of single domain antibodies onto the surface of the substrate.

In certain embodiments, the immunosensing substrate is the working electrode of an immunosensing device. The device may further comprise a counter electrode and a reference electrode.

In some embodiments, the conductive electrode is optionally configured with an array of interdigitated electrodes (IDEs). In some embodiments, the patterned electrode is electrochemically cleaned prior to any surface modification.

An exemplary configuration of IDEs comprises a plurality of parallel microband array electrodes, with each set of electrodes potentiostated individually. As such, one electrode is held at a potential to drive an oxidation reaction, while the adjacent electrode is held at a potential to drive a reduction reaction. Electroactive species generated at one electrode can diffuse across a small gap, with a width typically on the order of several microns, and are subsequently converted back to their original charge. This cyclic exchange of charges between two adjacent microelectrodes fabricated in an interdigitated pattern can greatly amplify the magnitude of the current output of the overall device. Advantageously, sensors comprising IDEs as the working electrode have improved detection sensitivity as a result of the increased current output generated by the microfabricated pattern (A. E. Cohen and R. R. Kunz, Sensors and Actuators B: Chemical, 62, 23 (2000); Y. Iwasaki and M. Morita, Current Separations, 14, 1 (1995)).

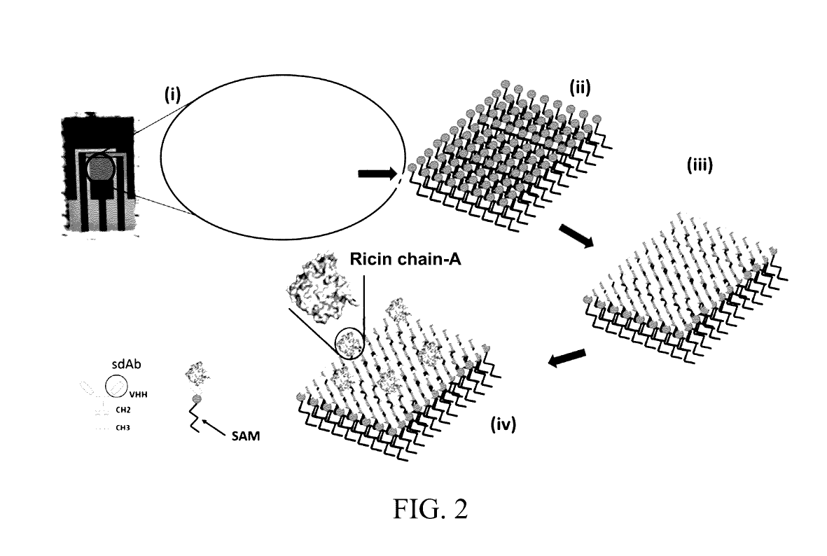

In some embodiments, the conductive electrode is chemically functionalized prior to the immobilization of the thermally-stable antibodies. In a preferred embodiment, the chemical functionalization comprises at least one self-assembled monolayer. The chemical functionalization provided herein is optionally included to facilitate the immobilization of the antibodies, as shown in, for example, FIG. 2.

Self-assembled monolayers (SAMs) are highly ordered molecular assemblies that form spontaneously by chemisorption of functionalized molecules on a variety of substrates such as metals, silicon, and glass. These molecules organize themselves laterally, most commonly via van der Waals interactions between long aliphatic chains. The thickness of a typical SAM is between about 10 Å and about 40 Å.

In an exemplary embodiment, the self-assembled monolayer comprises a cross-linking reagent, such as dithiobis(succinimidyl propionate) (DTSP), capable of reacting with both the amino groups of the antibodies and the electrode. DTSP covalently binds with the surface of the metal electrode, effectively rendering the surface ready to be modified with the antibodies. In some embodiments, the binding of DTSP-SAM to the surface of the electrode is accomplished by reducing the DTSP solution with sodium borohydride (NaBH₄).

In some embodiments, the antibodies are single-domain antibodies. Preferred embodiments provide that the single-domain antibodies are derived from species belonging to the Camelidae family.

Single-domain antibodies are antibodies whose complementary determining regions are part of a single-domain polypeptide. Examples of single-domain antibodies include, but are not limited to, heavy chain antibodies, antibodies naturally devoid of light chains, single domain antibodies derived from conventional 4-chain antibodies, and recombinant antibodies. The single-domain antibody provided herein is a naturally occurring single-domain antibody known as heavy chain antibody devoid of light chains, and is referred to as a VHH to distinguish it from the conventional VH of four-chain immunoglobulins.

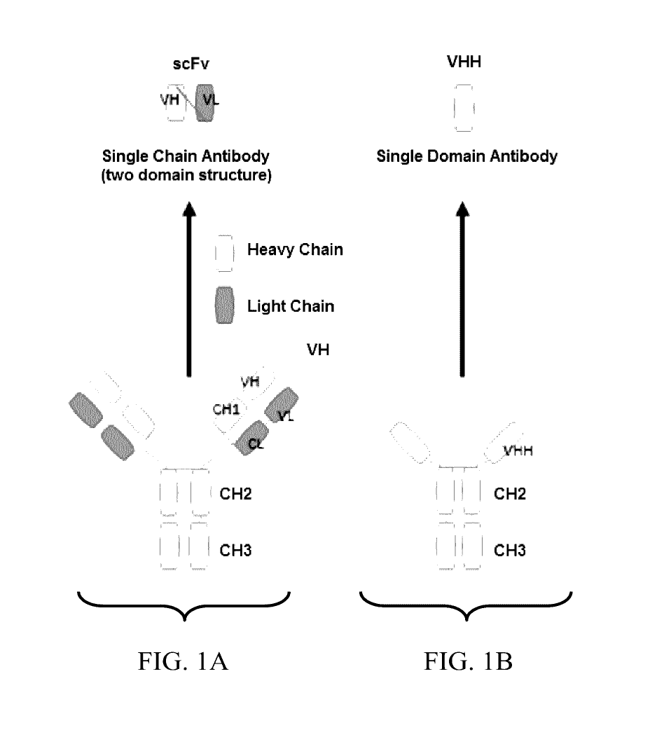

The terms “camelid-derived antibody” or “camelid-derived VHH” are used interchangeably herein and refer to antibodies obtained from members of the Camelidae family that include, but are not limited to, camel species (Camelus bactrianus and Camelus dormaderius), llama species (Lama paccos, Lama glama, and Lama vicugna), alpaca species (Vicugna pacos), guanaco species (Lama guanicoe), and vicuna species (Vicugna vicugna). Other species besides Camelidae may also produce heavy chain antibodies naturally devoid of a light chain; such VHHs are within the scope of the subject invention. In contrast to a conventional single chain antibody (FIG. 1A), FIG. 1B shows the structure of a VHH and its binding fragment.

A camelid-derived VHH has a molecular weight approximately one-tenth of that of a human IgG molecule. These antibodies have a physical diameter of only a few nanometers, allowing them to bind to antigenic sites that are functionally invisible to larger antibody proteins. The low molecular weight and compact size make the antibodies resistant to extreme pH and to proteolytic digestion. As with other antibodies of non-human origin, an amino acid sequence of a camelid derived antibody can be altered recombinantly to obtain a sequence that more closely resembles a human sequence, i.e., the antibody can be “humanized”.

In another aspect, the subject invention provides a method of detecting a target antigen, comprising:

a) providing an immunosensing substrate, the surface of the substrate being configured to comprise an array of interdigitated electrodes and chemically immobilized with a layer of antibodies;

b) contacting the substrate with a sample;

c) applying a voltage to the substrate; and

d) monitoring a change in current response of the substrate as antigens bind with the immobilized antibodies.

In certain embodiments, the substrate is the working electrode of an immunosensing device, which may further comprise a counter electrode and a reference electrode.

In certain embodiments, the conductive electrode is chemically functionalized by, for example, at least one self-assembled monolayer prior to the immobilization of the antibodies.

In a preferred embodiment, the single-domain antibodies are derived from species of the Camelidae family.

In some embodiments of the invention, the electrochemical response of the device is measured by monitoring the redox current output of the electrode as a function of varying the applied voltage, a technique known as cyclic voltammetry (CV). In certain embodiments, a reduction-oxidation (redox) probing moiety is employed, and preferably comprises a combination of ferricyanide and ferrocyanide anions in salt forms such as, for example, potassium salts.

Cyclic voltammetry is a type of potentiodynamic electrochemical measurement that can be used to evaluate the electrochemical properties of an analyte in solution. CV generally involves ramping the potential of a working electrode linearly versus time up to a certain set potential, at which point the direction of potential ramping is reversed. A plurality of cyclic potential ramps can be applied to the electrode during a single measurement. The current at the conductive electrode can be plotted versus the applied voltage to give a cyclic voltammogram trace.

In addition to measuring the current response of the substrate, in some embodiments of the subject invention CV is used to electrochemically treat the surface of the electrode. Specifically, CV can be used to roughen the surface of the electrode and, thus, increase its contact area with the subsequently adsorbed SAMs and/or antibodies.

Other analytical methods are also available for measuring the electrochemical response of the electrode. Non-limiting examples include chronoamperometry, chronovoltammetry, and differential pulse voltammetry. Persons of ordinary skill in the art would recognize that other suitable electrochemical techniques, now known or hereafter developed, can also be employed to detect a target antigen using the immunosensing devices provided herein.

A redox probing moiety is a labelling molecule that undergoes a reversible redox reaction without using oxygen as an electron mediator. In addition to the preferred embodiments provided herein, non-limiting examples of redox probing moieties include members of the quinone family, such as hydroxyquinone and anthraquinone. The redox probing moiety can be immobilized onto the surface of the electrode. Alternatively, as provided herein, the redox probing moiety is dissolved in a physiologically compatible buffer solution, a non-limiting example being phosphate buffer solution (PBS).

The sample can be, for example, human physiological fluids, cell cultures, food samples, and environmental samples. In specific embodiments, the sample is a human physiological fluid selected from blood, plasma, serum, saliva, urine, and tears.

According to certain embodiments, the conductive substrate is contacted with the sample prior to being subjected to an electrochemical treatment such as CV. Any suitable method for contacting the substrate with a sample may be used. For example, suitable methods include rinsing, dipping, or immersing the substrate in a sample, passing a stream comprising the sample over the substrate, or a combination thereof.

In a preferred embodiment, the sample is placed in contact with the substrate for about 30 minutes. Any known method of contacting a substrate with a sample can be adapted for use in the present invention, and those skilled in the art would recognize that the duration of immersion can be varied within a reasonable range.

In some embodiments, the application of voltage to the electrode is cyclic, ranging from about −1.0 V to about 1.0 V. Exemplary embodiments provide that the voltage is cycled between about −0.6 V to about 0.6 V.

Embodiments of the subject invention provide that the performance of the immunosensing substrate, evaluated as the change in current response to the applied voltage in comparison with a neat substrate, remains effectively constant for 1 day, 2 days, 3 days, 4 days, 5 days, 6 days, 7 days or more, at temperatures between room temperature and about 40° C. or higher. In a particular embodiment, the performance of the immunosensing substrate decreases by about 16% per day for up to about 3 days at temperatures between room temperature and about 55° C. “Room temperature” as used herein is between about 15° C. and about 25° C.

In an exemplary embodiment, the target antigen is ricin-chain A, derived from the castor oil plant Ricinus communis. Ricin is a naturally-occurring toxin and is extremely toxic to humans.

Ricin is a member of the type II ribosome inactivating protein (RIP) family of toxins. Having a molecular weight of about 65 kDa, the ricin toxin comprises two dissimilar polypeptide chains, chain A and chain B, held together by a disulfide bond. Chain A is an RNA N-glycosidase that depurinates a conserved adenosine residue located within the sacrin/ricin loop of the eukaryotic 28s ribosomal RNA (rRNA). Depurination of this residue results in an immediate cessation of ribosome progression, which subsequently inhibits protein synthesis. Chain B binds with micromolar affinity to α(1-3)-linked galactose and N-acetylgalactosamine receptors that are expressed on the surface of all mammalian cell types. Binding of the chain B to these receptors mediates internalization and retrograde transport of the ricin toxin to the endoplasmic reticulum (ER). In the ER, chain A dissociates from chain B and is then retrotranslocated across the ER membrane into the cytosol where it gains access to rRNA targets.

With ricin-chain A measuring approximately 32 kDa in molecular weight, better detection and binding affinity is achieved by utilizing small antibodies such as VHH (approximately 15 kDa in molecular weight) as the sensing angent, rather than the conventional single-chain antibodies whose sizes are in the range of 150 kDa. As a result, VHH antibodies can be employed to detect small antigens with improved stability against changes in environmental conditions such as temperature and pH.

In yet another aspect, the subject invention provides an electrochemical immunosensing substrate, whose surface is configured to comprise an array of interdigitated electrodes and chemically immobilized with a layer of antibodies.

In certain embodiments, the immunosensing substrate is the working electrode of an immunosensing device, which may further comprise a counter electrode and a reference electrode.

In some embodiments, devices comprising the immunosensing substrate provided herein can be worn. Advantageously, these devices can function for extended periods of time and even in harsh environments.

In certain embodiments, the working electrode is optionally functionalized by at least one self-assembled monolayer prior to the immobilization of the antibodies.

According to a preferred embodiment, the single-domain antibodies are derived from members of the Camelidae family.

In an exemplary embodiment, the device is stable at temperatures between room temperature and about 40° C. for up to about 7 days.

### EXAMPLES

The following are examples that illustrate the aforementioned embodiments and should not be construed as limiting. All of the chemical supplies provided herein, unless otherwise noted, were obtained via commercial sources and are readily available for procurement.

### Example 1

Interdigitated microelectrode (IDE) of 10 mm width and electrode gapof10 mm was used for fabrication of the immunosensor. IDEs were patterned on 4″ oxidized silicon wafers by traditional microfabrication processes (FIG. 2). First, MicroChem AZs 5214E photoresist was spin-coated on the oxidized wafer, and the IDE pattern was exposed using OA1800 Mask Aligner. After development in AZs MIF 300, chromium (20 nm) and gold (200 nm) were deposited via electron beam evaporation. The micro IDEs were obtained after a lift-off process. MicroChem SU-8 photoresist was then patterned to form wells above the IDEs for a fixed volume of approximately 5 μL.

Prior to the DTSP-SAM modification, the IDEs were cleaned using approximately 5 μL of H₂SO₄ (about 0.1 M) under CV as a function of varying applied voltage at a scan rate of about 50 mV/s. The parameters of the cleaning process were optimized using the aforementioned CV technique with about 5 μL of PBS (pH≈7.4) containing about 5 mM of a mixture of potassium ferrocyanide and potassium ferricyanide (K₃[Fe(CN)₆]/K₄[Fe(CN)₆]) at a scan rate of approximately 50 mV/s. The magnitude of the current response obtained was found to increase beginning with 5 scanning cycles and up to 10 scanning cycles. Higher current response was observed in the case where the IDEs were cleaned using 9 cycles and higher. However, the electrode started to etch after 9 scanning cycles. Therefore, the IDEs were cleaned using 7 scanning cycles in about 5 μL of H₂SO₄ (approximately 0.1 M). All IDEs were cleaned using the same process and exhibited similar current response without morphological damage.

### Example 2

Ricin_{sdAb} was obtained from the Center for BioMolecular Science and Engineering, Naval Research Laboratory. Ricin Chain-A antigen were obtained from BEI Resources. Dithiobis (succinimidyl propionate) (DTSP), sodium borohydride (NaBH4), and all other chemicals were purchased from Sigma-Aldrich. Each chemical was used as-is, without any further purification.

For the fabrication of the DTSP-SAM modified IDEs, a solution of DTSP (about 2 mg/mL in acetone) was prepared and subsequently reduced using NaBH₄ (about 10 mg/mL in deionized (DI) water). The IDEs were then immersed in the reduced DTSP solution for approximately 2 hours. After SAM modification, all IDEs were rinsed with acetone and then with DI water to remove any unbound DTSP molecules.

About 5 μL of ricin_{sdAbs} was covalently immobilized onto the modified IDEs' surface via a facile reaction between the amino group of the antibody and the reactive succinimidyl group present at the surface of the DTSP-SAM. The DTSP-SAM/IDEs were incubated with a sample of ricin_{sdAbs} for approximately 2 hours followed by carefully washing the IDEs with isopropyl alcohol (IPA) and DI water to remove any unbound molecules.

### Example 3

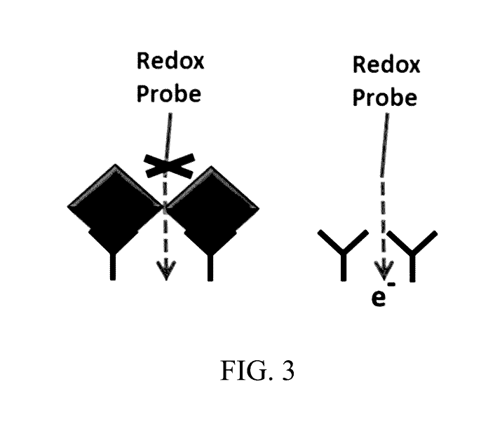

The fabricated ricin_{sdAbs}/DTSP-SAM/IDEs immunoelectrodes were used to detect various concentrations of ricin-chain A using the CV techniques disclosed in Example 1, wherein the modified electrodes were immersed in about 5 μL of PBS (pH is approximately 7.4) comprising approximately 5 mM of K₃[Fe(CN)₆]/K₄[Fe(CN)₆]. The electrochemical and immunochemical reaction at the surface of the IDEs is shown in FIG. 3.

About 5 μL of ricin-chain A antigen was placed in contact with the electrochemical immunosensor. The antigen sample was incubated for 30 minutes on the immunodelectrodes to ensure proper binding. The IDEs were then washed with IPA and DI water to remove any unbound antigens.

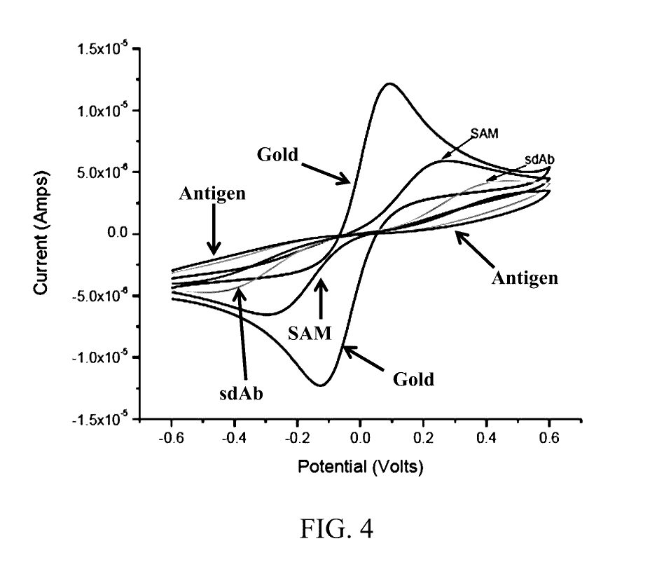

As shown in FIG. 4, the result of the CV studies indicates that the magnitude of current response decreases upon the onset adsorption of ricin_{sdAbs} and binding between ricin_{sdAbs} and ricin-chain A, both due to the formation of an insulating immuno-complex that acts to inhibit the electron transport from the electrolyte to the surface of the IDEs.

### Example 4

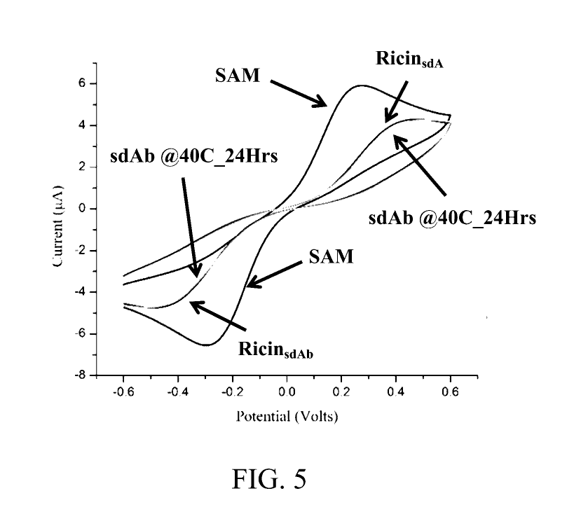

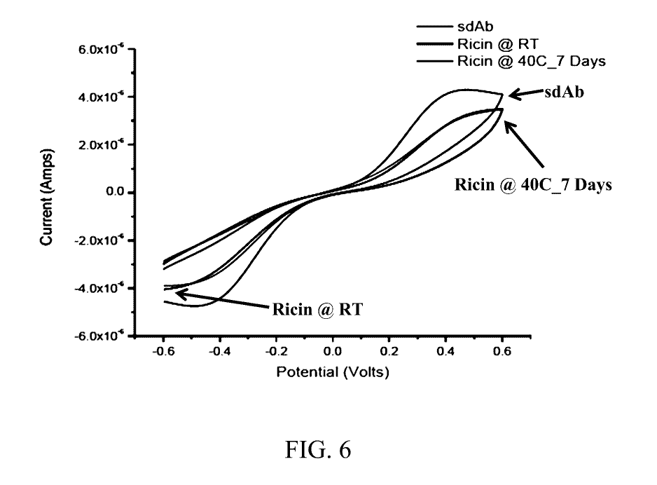

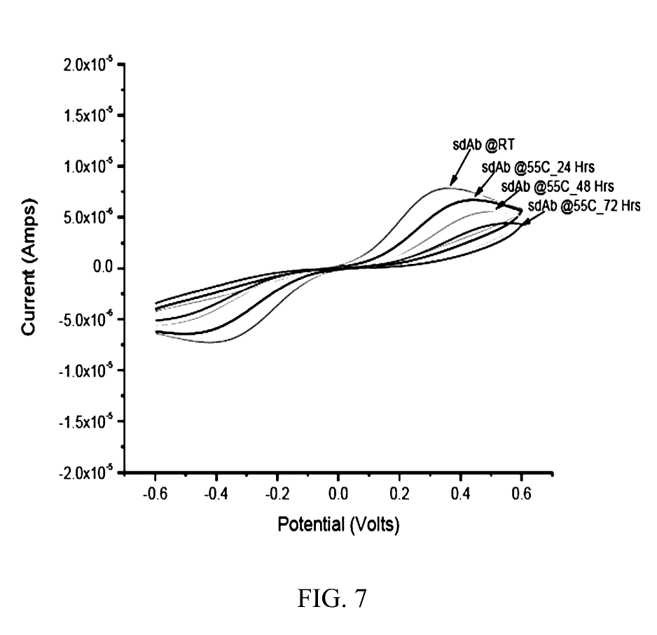

A CV study was also carried out to study the shelf-life of the ricin_{sdAb}/DTSP-SAM/IDEs at intervals of about 24 hours at room temperature, at about 40° C., and at about 55° C., respectively. The sdAbs were found to be stable at approximately 40° C. when compared to monoclonal antibodies, which degraded at RT within about 24 hours. CV studies of the immunosensor stored at approximately 40° C. after about 24 hours (FIG. 5) demonstrated that virtually constant current response was observed as time elapsed, suggesting that the antibody layer was stable at about 40° C. FIG. 6 confirms the stability of the antigen-bound immunosensors for about 7 days at approximately 40° C. FIG. 7 demonstrates that the stability of the immunosensor stored at about 55° C. is compromised by approximately 16% per day for up to 3 days.

### Example 5

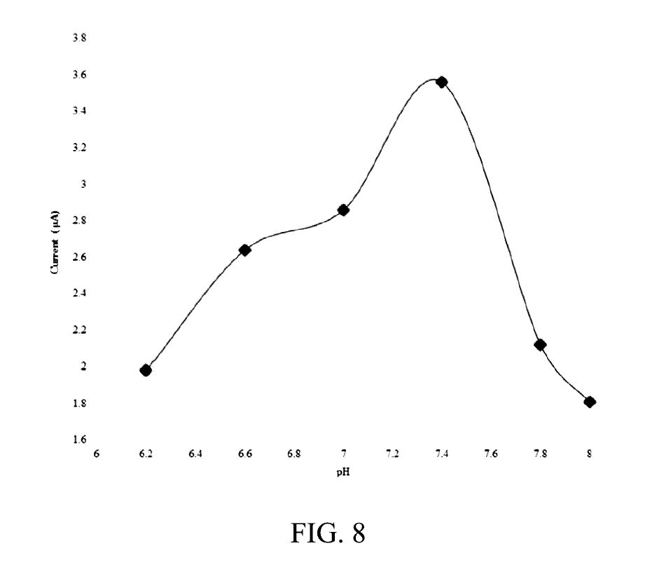

The influence of pH on the electrochemical responses of an immunosensor was evaluated. The immunosensors were tested by cyclic voltammetry in the presence of a series of PBS having pH ranging from 6.2 to 8 using K₃[Fe(CN)₆]/K₄[Fe(CN)₆] as the redox probe. The experimental results showed that an increase in pH from 6.2 to 7.4 resulted in an increased peak current. Further increment in pH resulted in the peak current decreasing. These results showed that the maximum current response occurred at pH 7.4 (FIG. 8). Therefore, a pH of 7.4 was maintained for all experiments. The decrease in current response at a pH above 7.4 can be attributed to the decreased biological activity of the antibody in strong alkaline solution (Chemicon International Inc., 1998. Introduction to Antibodies. 2nd ed., Chemicon International Inc).

### Example 6

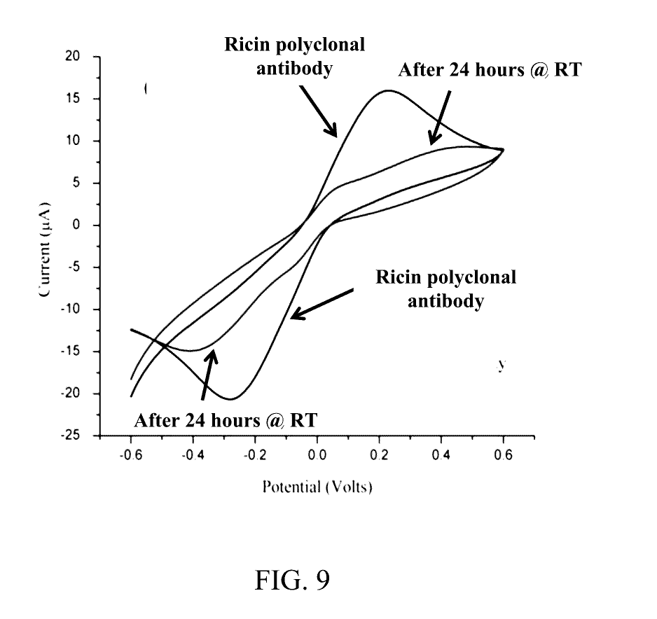

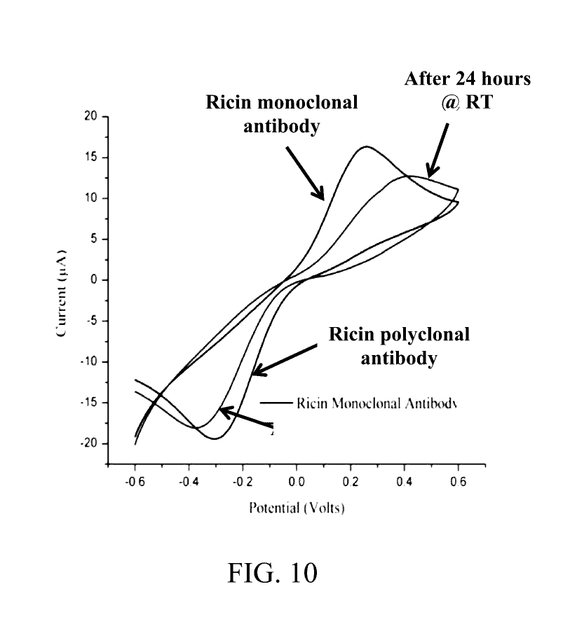

CV experiments were performed for ricin polyclonal antibodies, monoclonal antibodies, and single-domain antibodies, respectively. The results from CV analysis indicated that the magnitude of current response measured by the redox probe K₃[Fe(CN)₆]/K₄[Fe(CN)₆] for ricin_{sdAb} (FIG. 4) was lower than that of both polyclonal antibodies (FIG. 9) and monoclonal antibodies (FIG. 10). This can be explained by the smaller sizes of the sdAbs. The diminutive size of sdAbs (MW approximately 2-15 kDa) (Eyer, L., Hruska, K., 2012. Single-domain antibody fragments derived from heavy-chain antibodies: a review. Vet. Med. 57 (9), 439-513.; Harmsen, M. M., DeHaard, H. J., 2007. Properties, production, and applications of camelid single-domain antibody fragments. Appl. Microbiol. Biotechnol. 77 (1), 13-22.) allows them to bind in spaces inaccessible to conventional monoclonal and polyclonal antibodies. Aptamers (MW approximately 5-15 kDa) (Lakhin, A. V., Tarantul, V. Z., Gening, L. V., 2013. Aptamers: problems, solution sand prospects. Acta Nat. 5(4), 34-43.) and scFvs (MW approximately 25 kDa) (Bird, R. E., Hardman, K. D., Jacobson, J. W., Johnson, S., Kaufman, B. M., Lee, S. M., Lee, T., Pope, S. H., Riordan, G. S., Whitlow, M., 1988. Single-chain antigen-binding proteins. Science, 242(4877), 423-426.) also have this advantage. Furthermore, the magnitude of CV response decreased on the addition of the ricin chain-A antigen (FIG. 4). This is because electron transport from electrolyte to the surface of the IDE was inhibited by the formation of insulating immuno-complex between ricin_{sdAb} and ricin chain-A antigen.

### Example 7

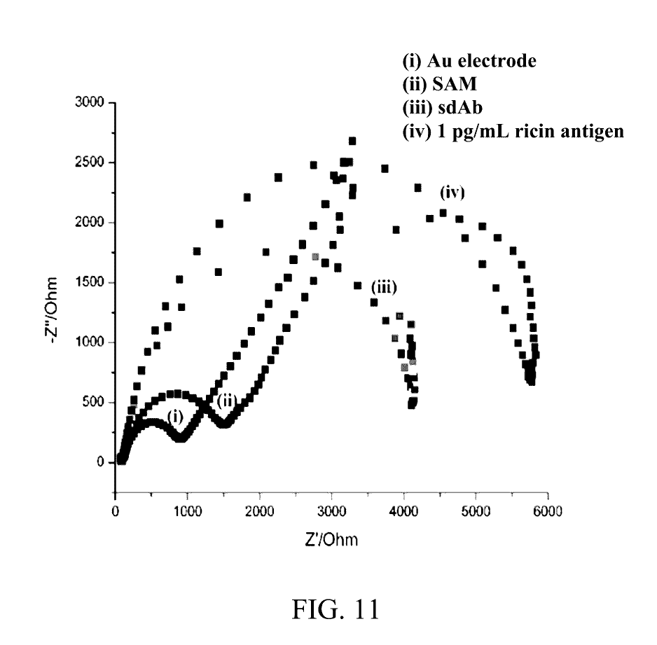

The morphology of the immunosensor was further characterized using electrochemical impedance spectroscopy (EIS). FIG. 11 shows the EIS measurements for each additional layer added onto the Au electrode: the SAM, the ricin_{sdAb}, and ricin chain-A antigen. The EIS measurements were performed in the presence of K₃[Fe(CN)₆]/K₄[Fe(CN)₆] redox probe. Further, CV measurements were performed. Advantageously, CV technique is easy to miniaturize and can be integrated with ultra-low-power microelectrics for POC applications. Previously, a micro-power potentiostat has been demonstrated for POC sensing applications (Cruz, A. F. D., Norena, N., Kaushik, A., Bhansali, S., 2014. A low-cost miniaturized potentiostat for point-of-care diagnosis. Biosens. Bioelectron. 62,2 49-254).

### Example 8

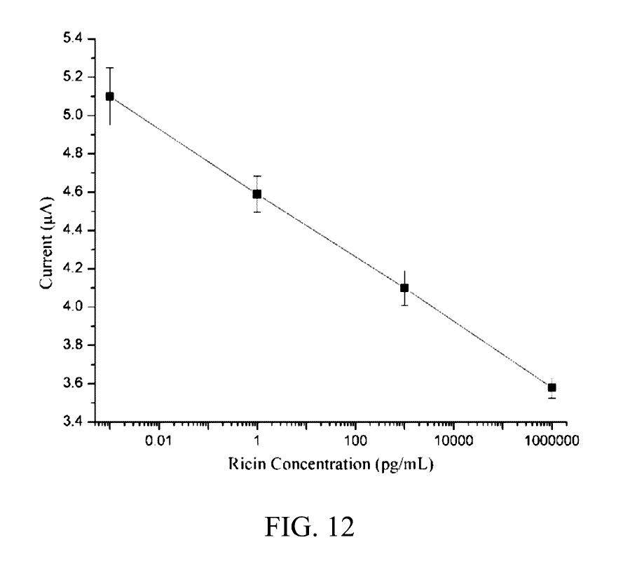

For further analysis, the magnitude of the current response for different concentrations (1 fg/mL-1 μg/mL) of ricin chain-A was measured via CV. The magnitude of current was found to decrease with increasing concentration by standard successive addition technique (FIG. 12). The detailed successive addition technique has been reported previously (Arya, S. K., Chornokur, G., Venugopal, M., Bhansali, S., 2010. Dithiobis(succinimidyl propionate) modified gold microarray electrode based electrochemical immunosensor for ultrasensitive detection of cortisol. Biosens. Bioelectron. 25 (10), 2296-2301.). Current magnitude vs. logarithm of the concentration of ricin chain-A antigen follows a linear relation of y=(3×10⁻⁶)−(7×10⁻⁸)×log (concentration of ricin chain-A antigen) from 1 log(fg/mL) to 1 log(μg/mL) with a detection limit of 1 log(fg/mL). The sdAbs-based immunosensor exhibits a sensitivity of 0.07 μA/log(g/mL)/cm² with a regression coefficient, R=0.999. It was observed that the current peak would saturate quickly at 1 log(μg/mL) or greater, which implies lesser binding sites on the sdAbs for ricin chain-A antigens. A limitation on the number of labels or tags that can be incorporated is also imposed by the small size of the sdAbs (Goldman, E., Liu, J., Bernstein, R., Swain, M., Mitchell, S., Anderson, G., 2009. Ricin detection using phage displayed single domain antibodies. Sensors, 9 (1), 542-555.); this, however, is not a limitation in this case due to the mechanism of the tag-free electrochemical sensing. Due to the less available binding sites, a lower concentration range was chosen for this experiment. Percentage change in peak current with 1 log(fg/mL) ricin chain-A from baseline value (only PBS without ricin chain-A) is 36%. From 1 log(fg/mL) to 1 log(pg/mL), 1 log(pg/mL) to 1 log(ng/mL) and 1 log(ng/mL) to 1 log(μg/mL) the percentage change in peak current was 7-10%. For further successive addition the change was much lower than the first and the current peak saturated quickly.

### Example 9

To study the thermal stability of the immunosensor, CV measurements were performed after heating the sensor at various temperatures. Initially thermal stability tests were carried out for SAM on IDEs to account for the background since SAM stability issues are uniquely defined by the bond between the film and substrate (Srisombat, L., Jamison, A. C., Lee, T. R., 2011. Stability: a key issue for self-assembled monolayers on gold as thin-film coatings and nanoparticle protectants. Colloids Surf. A: Physicochem. Eng. Asp. 390 (1-3), 1-19.). Short chain (C₃-C₅; where Cₙ=number of CH₂ bonds in CH₃-Cₙ—SH) thiol based SAM on gold substrate has been reported for desorption at 363 K (89.95° C.) (Vericat, C., Vela, M. E., Benitez, G., Carro, P., Salvarezza, R. C., 2010. Self-assembled monolayers of thiols and dithiols on gold: new challenges for a well-known system. Chem. Soc. Rev. 39 (5), 1805-1834.). Therefore, the temperature was varied from 25° C. to 80° C. on a single sensor. The sensor was placed on a hot plate and heated for 15 min prior to performing CV scans. Since the CV scans were taken in the presence of K₃[Fe(CN)₆]/K₄[Fe(CN)₆], the sensor was washed with DI water and dried with nitrogen before the next temperature treatment. A shift was observed in the current peak potential at 50° C., indicating a possible disorientation and blockage resulting in more negative reduction potentials. The lower temperature stability of SAM as compared to the ones reported in literature can be explained by the short 2-hour immersion time for SAM formation.

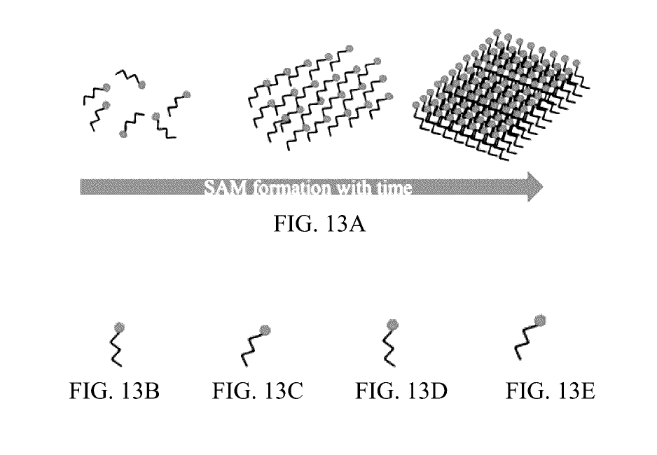

Through scanning tunneling microscopy (STM), the time-dependent organization of SAM on gold surfaces has been studied (Li, S.-S., Xu, L.-P., Wan, L. J., Wang, S.-T., Jiang, L., 2006. Time-dependent organization and wettability of decanethiol self-assembled monolayer on Au (111) investigated with STM. J. Phys. Chem. B, 110 (4), 1794-1799), and the time-dependent organization of thiols can be depicted as shown in FIGS. 13A-13C. A short immersion time of two hours is sufficient for a full coverage but with lesser density and more disorder. Conversely, a densely packed crystalline SAM monolayer is formed during prolonged overnight immersion, or for at least 24 hours, as commonly reported in literature. Here, a shorter SAM formation time of 2 hours was used because the current response, due to redox reactions between the electrode and antibody, shows a strong increase in the current with a decrease in the structural integrity (density and disorder or tilt) of the SAM. It should be noted that the current peak reduces in the case of a densely packed crystalline SAM due to charge screening. However, it is easy to fine tune the sensitivity of a sensor with densely packed SAM.

### Example 10

Further, a CV measurement was carried out for sensors immobilized with ricin_{SdAb} (ricin_{sdAb}/DTSP-SAM/IDEs). The temperature was varied from 25° C. to 50° C. in increments of 5° C. on a single sensor. Similar to the steps mentioned herein, the sensor was heated, measured, washed, and dried between measurements. The data obtained illustrate that the sdAbs-based electrochemical sensors were stable up to 40° C. (FIG. 5) when compared to monoclonal and polyclonal ricin antibodies, respectively, which degraded at room temperature within 24 hours (FIGS. 9 and 10). The redox current peaks decreased gradually from 45° C. onward and the peak potentials increased. One possible explanation for the increased instability is the gradual increase in the disorder of SAM with rising temperature. A disordered SAM tends to incline more toward the electrode surface unlike an ordered SAM which is extended normal to the electrode surface. An increased disorder in SAM implies that there is more coverage of the electrode surface, hence a decrease in the current magnitude. This type of blockage to the sensor surface because of an inclined SAM layer is depicted in FIG. 13C. Another possible explanation of the blockage to the sensor surface is the tilted orientation of the sdAbs which are immobilized on the SAM. This is depicted by FIG. 13D, where the sdAbs change their orientation with rise in temperature and subsequently block the surface of the electrode. A combination of both the disorder of SAM and the sdAbs' tilted orientation can also be a possibility as depicted by FIG. 13E.

Based on the aforementioned scenarios, four possible results of detection can be obtained by the immunosensor:
1. True positive: No change in redox current peaks for temperatures up to 40° C.
2. True negative: Reduction in redox current peaks for temperatures greater than 40° C.
3. False positive: Increase or no reduction in redox current peaks for temperatures greater than 40° C.
4. False negative: Reduction in redox current peaks for temperatures below 40° C.

The sdAb-immobilized immunosensors that exhibited the true positive nature also demonstrated true negative result; hence the redox current peaks in these sensors were reduced for temperatures greater than 40° C. None of the sensors showed a false positive because the time allowed for the formation of the SAM did not exceed 2 hours. In the case of a dense SAM, the structural integrity of the layer would be maintained and no disorder would be observed. However, blockage to the sensor surface can still be present due to changes in sdAbs' orientation with temperature. Similarly, the false negative nature of the few sensors can be attributed to the poorly formed SAMs.

For POC and wearable device applications, especially in remote areas, shelf-life of the sensor is crucial for inexpensive methods of transportation and long-term usage. The CV response in FIGS. 9 and 10 indicates that monoclonal and polyclonal antibodies are not stable at room temperature in a 24-hour period, rendering them with limited applications as components used in electrochemical immunosensors. However, the sdAb immunosensor demonstrated a shelf-life of at least 1 week (FIG. 6) after being heated to 40° C. as the maximum temperature. The CV response of the sdAbs-based immunosensors with bound ricin chain-A antigens remains the same after at least 7 days, confirming the unaltered condition of the immunosensor during the week. As mentioned earlier, the stability of a sensor is influenced by the stability of the SAM, the stability of the antibodies, and their respective binding efficiency, which together determined the extent of shelf-life of these immunosensors.

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

All patents, patent applications, provisional applications, and publications referred to or cited herein (including those in the “References” section) are incorporated by reference in their entirety, including all figures and tables, to the extent they are not inconsistent with the explicit teachings of this specification.

### REFERENCES

Chemicon International Inc., 1998. Introduction to Antibodies. 2nd ed., Chemicon International Inc.
Pierce Biotechnology Inc., 2005. Protein Stability and Storage, Pierce Biotechnol. Inc., p. 3.
Anderson, G. P., Bernstein, R. D., Swain, M. D., Zabetakis, D., Goldman, E. R., 2010. Binding kinetics of antiricin single domain antibodies and improved detection using a B chain specific binder. Anal. Chem. 82 (17), 7202-7207.
Anderson, G. P., Liu, J. L., Hale, M. L., Bernstein, R. D., Moore, M., Swain, M. D., Goldman, E. R., 2008. Development of antiricin single domain antibodies toward detection and therapeutic reagents. Anal. Chem. 80 (24), 9604-9611.
Arbabi Ghahroudi, M., Desmyter, A., Wyns, L., Hamers, R., Muyldermans, S., 1997. Selection and identification of single domain antibody fragments from camel heavy-chain antibodies. FEBS Lett. 414 (3), 521-526.
Arya, S. K., Chornokur, G., Venugopal, M., Bhansali, S., 2010. Dithiobis(succinimidyl propionate) modified gold microarray electrode based electrochemical immunosensor for ultrasensitive detection of cortisol. Biosens. Bioelectron. 25 (10), 2296-2301.
Bird, R. E., Hardman, K. D., Jacobson, J. W., Johnson, S., Kaufman, B. M., Lee, S. M., Lee, T., Pope, S. H., Riordan, G. S., Whitlow, M., 1988. Single-chain antigen-binding proteins. Science 242 (4877), 423-426.
Brinker, C. J., Ashley, C. S., Bhatia, R., Singh, A. K., 2002. Sol-gel method for encapsulating molecules, Google Patients.
Campuzano, S., Salema, V., Moreno-Guzman, M., Gamella, M., Yaliez-Sederio, P., Fernandez, L. A., Pingarrón, J. M., 2014. Disposable amperometric magnetoimmunosensors using nanobodies as biorecognition element. Determination of fibrinogen in plasma. Biosens. Bioelectron. 52, 255-260.
Chames, P., Van Regenmortel, M., Weiss, E., Baty, D., 2009. Therapeutic antibodies: successes, limitations and hopes for the future. Br. J. Pharmacol. 157 (2), 220-233.
Cruz, A. F. D., Norena, N., Kaushik, A., Bhansali, S., 2014. A low-cost miniaturized potentiostat for point-of-care diagnosis. Biosens. Bioelectron. 62, 249-254.
de Marco, A., 2011. Biotechnological applications of recombinant single-domain antibody fragments. Microb. Cell Factories 10, 44 44.
Even-Desrumeaux, K., Baty, D., Chames, P., 2010. Strong and oriented immobiliza-tion of single domain antibodies from crude bacterial lysates for high-throughput compatible cost-effective antibody array generation. Mol. Biosyst. 6 (11), 2241-2248.
Eyer, L., Hruska, K., 2012. Single-domain antibody fragments derived from heavy-chain antibodies: a review. Vet. Med. 57 (9), 439-513.
Goldman, E., Liu, J., Bernstein, R., Swain, M., Mitchell, S., Anderson, G., 2009. Ricin detection using phage displayed single domain antibodies. Sensors 9 (1), 542-555.
Goldman, E. R., Anderson, G. P., Conway, J., Sherwood, L. J., Fech, M., Vo, B., Liu, J. L., Hayhurst, A., 2008. Thermostable llama single domain antibodies for detection of botulinum A neurotoxin complex. Anal. Chem. 80 (22), 8583-8591.
Goldman, E. R., Anderson, G. P., Liu, J. L., Delehanty, J. B., Sherwood, L. J., Osborn, L. E., Cummins, L. B., Hayhurst, A., 2006. Facile generation of heat stable antiviral and antitoxin single domain antibodies from a semi-synthetic llama library. Anal. Chem. 78 (24), 8245-8255.
Graef, R. R., Anderson, G. P., Doyle, K. A., Zabetakis, D., Sutton, F. N., Liu, J. L., Serrano-González, J., Goldman, E. R., Cooper, L. A., 2011. Isolation of a highly thermal stable lama single domain antibody specific for Staphylococcus aureus en-terotoxin B. BMC Biotechnol. 11 (1), 86.
Hamers-Casterman, C., Atarhouch, T., Muyldermans, S., Robinson, G., Hammers, C., Bajyana Songa, E., Bendahman, N., Hammers, R., 1993. Naturally occurring antibodies devoid of light chains. Nature 363, 446-448.
Harmsen, M. M., De Haard, H. J., 2007. Properties, production, and applications of camelid single-domain antibody fragments. Appl. Microbiol. Biotechnol. 77 (1), 13-22.
Huang, L., Reekmans, G., Saerens, D., Friedt, J.-M., Frederix, F., Francis, L., Muylder-mans, S., Campitelli, A., Hoof, C. V., 2005. Prostate-specific antigen im-munosensing based on mixed self-assembled monolayers, camel antibodies and colloidal gold enhanced sandwich assays. Biosens. Bioelectron. 21 (3), 483-490.
Jayasena, S. D., 1999. Aptamers: an emerging class of molecules that rival antibodies in diagnostics. Clin. Chem. 45 (9), 1628-1650.
Kaushik, A., Arya, S. K., Vasudev, A., Bhansali, S., 2013. Nanocomposites based on chitosan-metal/metal oxides hybrids for biosensors applications. J. Nanosci. Lett. 3 (32), 18.
Lakhin, A. V., Tarantul, V. Z., Gening, L. V., 2013. Aptamers: problems, solutions and prospects. Acta Nat. 5 (4), 34-43.
Lan, E. H., Dunn, B., Zink, J. I., 2000. Sol-gel encapsulated anti-trinitrotoluene antibodies in immunoassays for TNT. Chem. Mater. 12 (7), 1874-1878.
Li, S.-S., Xu, L.-P., Wan, L.-J., Wang, S.-T., Jiang, L., 2006. Time-dependent organiza-tion and wettability of decanethiol self-assembled monolayer on Au(111) in-vestigated with STM. J. Phys. Chem. B 110 (4), 1794-1799.
Lillehoj, P. B., Huang, M.-C., Truong, N., Ho, C.-M., 2013. Rapid electrochemical detection on a mobile phone. Lab Chip 13 (15), 2950-2955.
Liu, J. L., Zabetakis, D., Lee, A. B., Goldman, E. R., Anderson, G. P., 2013. Single domain antibody-alkaline phosphatase fusion proteins for antigen detection—analysis of affinity and thermal stability of single domain antibody. J. Immunol. Methods 393 (1-2), 1-7.
Loncaric, C., Tang, Y., Ho, C., Parameswaran, M. A., Yu, H.-Z., 2012. A USB-based electrochemical biosensor prototype for point-of-care diagnosis. Sens. Actua-tors B: Chem. 161 (1), 908-913.
Maass, D. R., Sepulveda, J., Pernthaner, A., Shoemaker, C. B., 2007. Alpaca (Lama pacos) as a convenient source of recombinant camelid heavy chain antibodies (VHHs). J. Immunol. Methods 324 (1-2), 13-25.
MacCraith, B. D., McDonagh, C. M., O'Keeffe, G., McEvoy, A. K., Butler, T., Sheridan, F. R., 1995. Sol-gel coatings for optical chemical sensors and biosensors. Sens. Actuators B: Chem. 29 (1-3), 51-57.
Mascini, M., 2008. Aptamers and their applications. Anal. Bioanal. Chem. 390 (4), 987-988.
Nelson, A. L., 2010. Antibody fragments: hope and hype. Monoclon. Antibodies (mAbs) 2 (1), 77-83.
Radi, A.-E., 2011. Electrochemical aptamer-based biosensors: recent advances and perspectives. Int. J. Electrochem. 2011, 17.
Saerens, D., Huang, L., Bonroy, K., Muyldermans, S., 2008. Antibody fragments as probe in biosensor development. Sensors 8 (8), 4669.
Slocik, J. M., Kim, S. N., Auvil, T., Goldman, E. R., Liu, J., Naik, R. R., 2010. Single domain antibody templated nanoparticle resistors for sensing. Biosens. Bioelectron. 25 (8), 1908-1913.
Song, K.-M., Lee, S., Ban, C., 2012. Aptamers and their biological applications. Sen-sors 12 (1), 612-631.
Srisombat, L., Jamison, A. C., Lee, T. R., 2011. Stability: a key issue for self-assembled monolayers on gold as thin-film coatings and nanoparticle protectants. Colloids Surf. A: Physicochem. Eng. Asp. 390 (1-3), 1-19.
Swed, A., Cordonnier, T., Fleury, F., Boury, F., 2014. Protein encapsulation into PLGA nanoparticles by a novel phase separation method using non-toxic solvents. J. Nanomed. Nanotechnol. 5,241.
van der Linden, R. H. J., Frenken, L. G. J., de Geus, B., Harmsen, M. M., Ruuls, R. C., Stok, W., de Ron, L., Wilson, S., Davis, P., Verrips, C. T., 1999. Comparison of physical chemical properties of llama VHH antibody fragments and mouse monoclonal antibodies. Biochim. Biophys. Acta (BBA): Protein Struct. Mol. Enzymol. 1431 (1), 37-46.
Vericat, C., Vela, M. E., Benitez, G., Carro, P., Salvarezza, R. C., 2010. Self-assembled monolayers of thiols and dithiols on gold: new challenges for a well-known system. Chem. Soc. Rev. 39 (5), 1805-1834.
Wan, Y., Su, Y., Zhu, X., Liu, G., Fan, C., 2013. Development of electrochemical immunosensors towards point of care diagnostics. Biosens. Bioelectron. 47 (0), 1-11.
Wang, T., Duan, Y., 2011. Probing the stability-limiting regions of an antibody sin-gle-chain variable fragment: a molecular dynamics simulation study. Protein Eng. Des. Sel. 24 (9), 649-657.
Wesolowski, J., Alzogaray, V., Reyelt, J., Unger, M., Juarez, K., Urrutia, M., Cauerhff, A., Danquah, W., Rissiek, B., Scheuplein, F., Schwarz, N., Adriouch, S., Boyer, O., Seman, M., Licea, A., Serreze, D. V., Goldbaum, F. A., Haag, F., Koch-Nolte, F., 2009. Single domain antibodies: promising experimental and therapeutic tools in infection and immunity. Med. Microbiol. Immunol. 198 (3), 157-174.
Willuda, J., Honegger, A., Waibel, R., Schubiger, P. A., Stahel, R., Zangemeister-Wittke, U., Pluckthun, A., 1999. High thermal stability is essential for tumor targeting of antibody fragments: engineering of a humanized anti-epithelial glycoprotein-2 (epithelial cell adhesion molecule) single-chain Fv fragment. Cancer Res. 59 (22), 5758-5767.
Worn, A., Pluckthun, A., 2001. Stability engineering of antibody single-chain Fv fragments. J. Mol. Biol. 305 (5), 989-1010.
Zhu, Z., Shi, L., Feng, H., Susan Zhou, H., 2015. Single domain antibody coated gold nanoparticles as enhancer for Clostridium difficile toxin detection by electro-chemical impedance immunosensors. Bioelectrochemistry 101, 153-158.

We claim:

1. A method of detecting a target antigen, comprising:
a) providing an immunosensing substrate, the surface of the substrate being configured to comprise an array of interdigitated electrodes and chemically immobilized with a layer of antibodies;
b) contacting the substrate with a sample;
c) applying a voltage to the substrate; and
d) monitoring a change in current response of the substrate as antigens bind with the immobilized antibodies.

2. The method according to claim 1, characterized in that the substrate is further functionalized with at least one self-assembled monolayer prior to the immobilization of the antibodies.

3. The method according to claim 1, characterized in that the antibodies are single-domain antibodies.

4. The method according to claim 3, characterized in that the single-domain antibodies are isolated from species of the Camelidae family.

5. The method according to claim 1, characterized in that the application of the voltage is cyclic, ranging between about −1.0 V and about 1.0 V.

6. The method according to claim 4, characterized in that the binding of the antigens to the antibodies is thermally stable at 40° C. for at least 24 hours.

7. The method according to claim 1, characterized in that the immunosensing substrate is worn by a person or animal.

8. The method, according to claim 1, wherein the antigen is ricin.

9. An electrochemical immunosensing substrate, the surface of the substrate being configured to comprise an array of interdigitated electrodes and chemically immobilized with a layer of single-domain antibodies.

10. The substrate according to claim 9, being the working electrode of an immunosensing device, the device further comprising a counter electrode and a reference electrode.

11. The substrate according to claim 9, characterized in that the surface of the substrate is further functionalized with at least one self-assembled monolayer prior to the chemical immobilization of the single domain antibodies.

12. The substrate according to claim 9, characterized in that the single domain antibodies are isolated from species of the Camelidae family.

13. The substrate according to claim 12, being thermally stable at temperatures up to about 40° C. for at least 24 hours.

14. A method of fabricating the electrochemical immunosensing substrate of claim 9, comprising configuring the surface of a conductive substrate to comprise an array of interdigitated electrodes and chemically immobilizing a layer of single-domain antibodies onto the surface of the substrate.

15. The method according to claim 14, characterized in that the substrate is the working electrode of an immunosensing device, the device further comprising a counter electrode and a reference electrode.

16. The method according to claim 14, further comprising functionalizing the surface of the substrate prior to chemically immobilizing the single-domain antibodies thereto.

17. The method according to claim 16, characterized in that the functionalization comprises chemically immobilizing at least one self-assembled monolayer.

18. The method according to claim 14, characterized in that the single-domain antibodies are isolated form species of the Camelidae family.

19. The method according to claim 18, characterized in that the substrate is thermally stable at temperatures up to about 40° C. for at least 24 hours and up to 7 days.

20. A method of detecting a target antigen, comprising:
a) providing an immunosensing substrate, the surface of the substrate being configured to comprise an array of interdigitated electrodes and chemically immobilized with a layer of single domain antibodies that are isolated from a species of the Camelidae family;
b) contacting the substrate with a sample;
c) applying a voltage to the substrate; and
d) monitoring a change in current response of the substrate as antigens bind with the immobilized antibodies;
characterized in that the binding of the antigens to the antibodies is thermally stable at 40° C. for at least 24 hours, and further characterized in that the target antigen is ricin.
